(19) United States
(12) Patent Application Publication
(10) Pub. No.: US 2013/0253485 A1
(43) Pub. Date: Sep. 26, 2013

(54) MEDICAL EXAMINATION SYSTEM

(71) Applicants: Jens Fehre, Hausen (DE); Andreas Limmer, Furth (DE); Ralf Nanke, Neunkirchen am Brand (DE); Manfred Sechser, Neusorg (DE)

(72) Inventors: Jens Fehre, Hausen (DE); Andreas Limmer, Furth (DE); Ralf Nanke, Neunkirchen am Brand (DE); Manfred Sechser, Neusorg (DE)

(73) Assignee: Siemens Aktiengesellschaft, Munchen (DE)

(21) Appl. No.: 13/615,292

(22) Filed: Sep. 13, 2012

(30) Foreign Application Priority Data
Sep. 14, 2011 (DE) 10 2011 082 680.7

(51) Int. Cl. A61B 18/26; H02P 29/04

(52) U.S. Cl. CPC A61B 18/26; H02P 29/04
USPC 606/2.5; 318/366

(57) ABSTRACT

A system includes at least one device that includes at least one device component. For a defined movement of the at least one device component, a movement axis and/or a movement direction may be selected by a selection element and may be identified by an activatable lighting element. The identified movement axis and/or the identified movement direction may be released by at least one actuation element, so that a desired movement of the at least one device component may be performed.

This application claims the benefit of DE 10 2011 082 680.7, filed on Sep. 14, 2011.

### BACKGROUND

The present embodiments relate to a medical examination system having at least one device that includes at least one device component.

In systems having a number of movement axes or degrees of freedom for movement, which movement axis is being controlled or braked or which brake is assigned to which movement axis is uniquely identified. In many instances, the movement axes are activated by a manual operating device that may be moved relatively freely in space, thereby predefining the movement directions.

Such a system may be embodied, for example, as a medical system.

A medical system with the product name “ARCADIS Varic” is known, for example, from the product information brochure “Streamlined workflow and outstanding image quality in the OR” published by Siemens AG, order reference A91SC-20002-2C1. The “ARCADIS Varic” system is a mobile medical system that includes a C-arm. At one end of the C-arm, an x-ray source is disposed, and at another end of the C-arm, an x-ray detector is disposed. The C-arm may be moved translationally in all three spatial directions as well as angularly and orbitally. The individual movement axes and/or individual movement directions of the C-arm are identified, as described on page 13 of the abovementioned product information brochure, by color-coded scales that match corresponding colors of the respective brake. Even with color-coded brakes and scales for every movement direction, the movement axis currently being controlled and whether the movement axis is being braked or moved by actuating an associated actuation key of a remote operating device or which brake is assigned to which movement axis may not be uniquely identified.

The assignment of the actuation keys on the operating surface of the manual operating device to the actual movement directions is not always intuitively obvious to the user.

SUMMARY AND DESCRIPTION

The present embodiments may obviate one or more drawbacks or limitations in the related art. For example, a system having at least one device, in which at least one device component has improved intuitive operability, is provided.

The system (e.g., a medical system) includes at least one device. The at least one device includes at least one device component. For a defined movement of the device component, a movement axis and/or a movement direction may be selected by a selection element and identified by an activatable lighting element. The identified movement axis and/or the identified movement direction may be released by at least one actuation element, so that a desired movement of the device component may be performed.

With the system, a two-stage release of a selected device component takes place. In a first release stage, a selection element (e.g., a capacitive sensor) is first used to select a movement axis and/or movement direction for a defined movement of the selected device component, and a lighting element (e.g., a light-emitting diode) is used to identify the movement axis and/or the movement direction accordingly. In a second release stage, the identified movement axis and/or the identified movement direction is released by at least one actuation element (e.g., mechanical switch) so that the released device component is moved by motor or may be moved manually in the desired movement direction and/or about the desired movement axis.

When the selection element is touched, one embodiment of the system allows unique assignment and identifiability of the selected movement axis or the selected movement direction. In other words, before the actual movement of the selected device component about the desired movement axis or in the desired movement direction, the movement axis or the movement direction is uniquely assigned or may be uniquely identified. Simple and unique identifiability of the state of the brakes is also provided. Movement in an incorrect direction is therefore reliably prevented.

Before the actual movement of the device component is executed, the combination of at least one selection element and at least one lighting element for displaying the selected movement axis and/or the selected movement direction (e.g., direction encoding before release) provides a high level of protection against incorrect operation.

The use of a lighting element provides that the movement axis used and/or the movement direction used may be reliably identified, for example, in a darkened operating room.

According to advantageous embodiments of the system, the device may be, for example, a C-arm unit, another x-ray device, a lithotripter, a support table, or a combination thereof.

A schematically illustrated exemplary embodiment of the system is described in more detail below with reference to the drawing without, however, being restricted thereto. In the illustrated exemplary embodiment, the system is configured as a medical system and therefore includes at least one medical device.

### BRIEF DESCRIPTION OF THE DRAWINGS

FIG. 1 shows a medical system having two medical devices 1 and 2.

### DETAILED DESCRIPTION OF THE DRAWINGS

A mobile C-arm unit 1 includes a device component in the form of, for example, an approximately arc-shaped C-arm 3. Disposed opposite one another on the C-arm 3 are an x-ray source 4 at one end and an x-ray detector 5 at another end. The C-arm 3 is supported such that the C-arm may be moved along a periphery in a holder 6. The holder 6 is also disposed on a horizontal guide 7 that is supported on a vertical column 8 such that the height of the horizontal guide 7 may be adjusted. The holder 6 may also be twisted in relation to the horizontal.

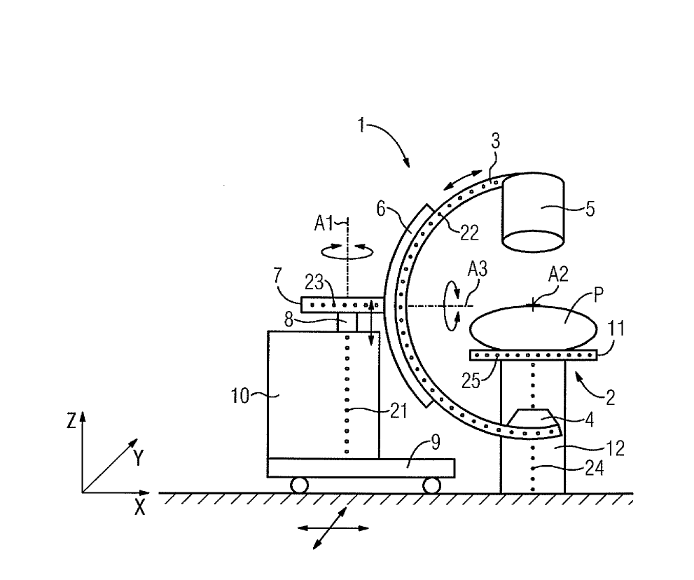

The vertical column 8 is connected in a guide (not shown in FIG. 1) to a frame 9 that also supports a device cabinet 10. The mobile C-arm unit 1 may therefore be easily brought to a patient support table 2.

The height of the C-arm 3 is adjusted, for example, by raising the C-arm 3 along a first axis A1. A rotational movement or pivoting movement of the C-arm 3 about the first axis A1 is also possible. An orbital rotation takes place about a second axis A2, and an angular rotation takes place about a third axis A3. Horizontal displacement of the C-arm 3 is achieved by moving the holder 6 in the x-direction.

Vertical adjustments, horizontal adjustments, rotational movements, pivoting movements, orbital rotations and angular rotations may be undertaken with the C-arm 3. These motions may be achieved using, for example, activatable electric motors in conjunction with position measurement units or manually. The electric motors and the position measurement units are not shown in the drawing for the sake of clarity.

With the illustrated C-arm unit 1, the movement axes and movement directions are identified by lighting elements. In the illustrated exemplary embodiment, a lighting element includes a number of light-emitting diodes. The lighting elements visible on the C-arm unit 1 are marked 21, 22 and 23.

The lighting element 21 identifies the height adjustment along the first axis A1, the lighting element 22 identifies the orbital rotation about the second axis A2, and the lighting element 23 identifies the horizontal adjustment in the x-direction.

For a defined movement of the C-arm 3 (e.g., device component), a movement axis or movement direction is selected by a selection element and identified by an activatable lighting element.

The selection element is disposed, for example, in a remote operating device but may also be disposed on the C-arm unit 1 within the scope of the present embodiments.

After the desired movement axis or the desired movement direction has been selected, the identified movement axis or the identified movement direction is released by at least one actuation element, so that a desired movement of the C-arm 3 (e.g., device component) may be performed.

The actuation element is, for example, also disposed in the remote operating device and may also be disposed on the C-arm unit 1 within the scope of the present embodiments.

With the C-arm unit 1 illustrated in the drawing, a two-stage release of the C-arm 3 (e.g., selected device component) takes place. In a first release stage, a selection element (e.g., a capacitive sensor) is first used to select a movement axis and/or a movement direction for a defined movement of the C-arm 3. A lighting element, which in the illustrated exemplary embodiment includes light-emitting diodes, is used to identify the movement axis and/or the movement direction. In a second release stage, the identified movement axis and/or the identified movement direction is released by at least one actuation element (e.g., a mechanical switch) so that the released C-arm 3 is moved in the desired movement direction and/or about the desired movement axis.

When the selection element is touched, the selection element provides unique assignment and identifiability of the selected movement axis or the selected movement direction. In other words, before the actual movement of the C-arm 3 about the desired movement axis or in the desired movement direction, the movement is uniquely assigned or may be uniquely identified. Simple and unique identifiability of the state of the brakes is also provided. Movement in an incorrect direction is therefore reliably prevented.

Before the actual movement of the C-arm 3 is executed, the combination of at least one selection element and at least one lighting element 21, 22, 23 for displaying the selected movement axis and/or the selected movement direction (e.g., direction encoding before release) provides a high level of protection against incorrect operation.

The use of at least one lighting element 21, 22, 23 provides that the movement axis used and/or the movement direction used may be reliably identified, for example, in a darkened operating room.

The selection elements (e.g., handles of the brakes of the C-arm 3) are provided with capacitive sensors, so that when the selection elements are touched, the lighting elements 21, 22, 23 on the corresponding axes A1, A2, A3 light up in the manner of a scale. The user may see whether he/she is addressing the correct axis A1, A2, A3, only releasing the brake using the actuation element (e.g., lever) if this is the case. In the illustrated exemplary embodiment, the lighting elements 21, 22, 23 light up differently in the braked state and during movement (e.g., flashing when the corresponding selection element is touched and constant to identify the scale when the movement is executed). Alternatively, only the non-braked axes may light up. In both instances, an operator may see immediately which axes may be moved.

The light-emitting diodes may also be matched in shape and/or color to the corresponding movement axes or movement directions (e.g., blue light-emitting diodes for orbital rotation, orange light-emitting diodes for angular rotation, green light-emitting diodes for horizontal adjustment and yellow light-emitting diodes for height adjustment of the C-arm 3).

The patient support table 2 illustrated in the drawing includes a device component in the form of a support surface 11 that is disposed on a column 12. The support surface 11, which serves to support a patient P during a medical examination, may be displaced relative to the column 12 in all spatial directions along the Cartesian coordinates X, Y, Z and may be tilted relative to the horizontal. These movements may also be made using electric motors in conjunction with position measurement units. Again, neither the electric motors nor the position measurement units are shown in the drawings for purposes of clarity.

With the illustrated patient support table 2, the movement axes and the movement directions are identified by lighting elements. Also, with the patient support table 2, each of the lighting elements includes a number of light-emitting diodes. The lighting elements visible on the patient support table 2 are marked 24 and 25.

The activatable lighting element 24 identifies a height adjustment of the support surface 11 parallel to the first axis A1, and the activatable lighting element 25 identifies a horizontal adjustment of the patient support table 2. The horizontal adjustment involves a displacement in the x-direction.

The disclosure relating to the two-stage release of the C-arm 3 applies in the same manner to the selection and identification of a movement axis and/or a movement direction for a defined movement of the support surface 11 (e.g., device component) and to the release of the identified movement axis or the identified movement direction for performing a movement.

In the illustrated exemplary embodiment, activation takes place via keys that form the selection elements and are disposed on a remote operating device (e.g., manual operating device). The keys are embodied with two stages (e.g., as capacitive switches for identifying when the key is touched and as mechanical switches for executing the actual movement of the support surface 11). In one embodiment, the selection elements may also be disposed on the patient support table 2.

The lighting elements 24, 25 disposed on the patient support table 2 also display to the user the (selected) movement direction (e.g., upward, downward, sideways). This prevents incorrect movements of the support surface 11 during interventions (e.g., operative or diagnostic interventions). The displaying of the selected movement direction offers a high level of safety, for example, if the orientation of the manual operating device does not correspond precisely to the Cartesian coordinate system of the support surface 11.

Even though the invention is illustrated and described in detail using the exemplary embodiment, the invention is not restricted by the exemplary embodiment illustrated in the drawing. Rather, the person skilled in the art may also derive other variants from this without departing from the underlying inventive concept.

As may be seen from the description of the exemplary embodiment illustrated in the drawing, the system of the present embodiments improves intuitive operability for at least one device component 3, 11 in at least one device 1, 2. In one embodiment, the system may be used in a medical system having at least one medical device 1, 2.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A system comprising:
at least one device comprising at least one device component;
a selection element operable to select a movement axis, a movement direction, or the movement axis and the movement direction for a defined movement of the device component;
an activatable lighting element operable to identify the movement axis, the movement direction, or the movement axis and the movement direction; and
an actuation element operable to release the identified movement axis, the identified movement direction, or the identified movement axis and the identified movement direction, so that a desired movement of the at least one device component is performable.

2. The system as claimed in claim 1, wherein the at least one device is configured as a medical device.

3. The system as claimed in claim 1, wherein the at least one device is configured as a C-arm unit.

4. The system as claimed in claim 1, wherein the at least one device is configured as an x-ray device.

5. The system as claimed in claim 1, wherein the at least one device is configured as a lithotripter.

6. The system as claimed in claim 1, wherein the at least one device is configured as a support table.

7. The system as claimed in claim 1, wherein the actuation element comprises a capacitive sensor.

8. The system as claimed in claim 1, wherein the lighting element comprises light-emitting diodes.

9. The system as claimed in claim 1, wherein the actuation element is configured as an electromechanical switch or a mechanical switch.

10. The system as claimed in claim 1, wherein the actuation element comprises a pneumatic switch.

11. The system as claimed in claim 1, wherein the actuation element comprises a hydraulic switch.

12. The system as claimed in claim 2, wherein the medical device is configured as a C-arm unit.

13. The system as claimed in claim 2, wherein the medical device is configured as an x-ray device.

14. The system as claimed in claim 2, wherein the medical device is configured as a lithotripter.

15. The system as claimed in claim 2, wherein the at least one device is configured as a support table.

16. The system as claimed in claim 2, wherein the actuation element comprises a capacitive sensor.

17. The system as claimed in claim 2, wherein the lighting element comprises light-emitting diodes.

18. The system as claimed in claim 7, wherein the actuation element is configured as an electromechanical switch or a mechanical switch.

19. The system as claimed in claim 7, wherein the actuation element comprises a pneumatic switch.

20. The system as claimed in claim 7, wherein the actuation element comprises a hydraulic switch.
